(19) United States
(12) Patent Application Publication
(10) Pub. No.: US 2021/0378880 A1
(43) Pub. Date: Dec. 9, 2021

(54) HEATED MENSTRUAL CUP

(71) Applicant: Abyan A. Nur, Hopkins, MN (US)

(72) Inventor: Abyan A. Nur, Hopkins, MN (US)

(21) Appl. No.: 17/339,087

(22) Filed: Jun. 4, 2021

Related U.S. Application Data
(60) Provisional application No. 63/035,106, filed on Jun. 5, 2020.

(51) Int. Cl. A61F 13/20; A61F 13/551; A61L 15/28; A61L 15/18; A61L 15/22

(52) U.S. Cl. CPC A61F 13/2045; A61F 13/55175; A61L 15/225; A61L 15/18; A61L 15/28

(57) ABSTRACT

A heated menstrual cup includes a main cup body comprising an upper section defining a substantially circular opening and a lower section comprising a fluid collection portion defining a closed lower end; and a flexible ring configured for removable attachment to the upper section of the flexible main cup body at the open upper end and further configured to be positioned around a cervix of a female human, the flexible ring defining a channel entirely enclosed therein. The flexible ring includes a sealed air container positioned within the channel, the air container containing air and comprising a frangible shell constructed to be broken to unseal the air container and release the air into the channel; and a thermogenic composition contained within the channel in isolation from air when the sealed air container is sealed, the thermogenic composition comprising one or more components that generate heat when in contact with the air released from the air container.

### CROSS-REFERENCE TO RELATED APPLICATIONS

The present application claims priority to U.S. Provisional Application Number which is incorporated herein by reference in its entirety.

### FIELD

The present disclosure relates to a device for relieving menstrual cramps and collecting menstrual fluid.

### BACKGROUND

Many women and girls worldwide suffer moderate to severe pain from premenstrual syndrome (PMS) and dysmenorrhea, typically a few days before to a few days after the start of their menstrual period, due to heavy cramping caused by uterine contractions. Women seeking relief from such cramping often turn to over-the-counter pain medication, such as ibuprofen or acetaminophen. However, the effectiveness of over-the-counter pain medication for menstrual cramps varies from individual to individual, to the extent it may not provide adequate pain relief for some individuals. Moreover, these medications may relieve pain for only a few hours before wearing off, necessitating additional doses, and may cause unwanted side effects and/or health risks. In addition, over-the-counter medications and other known means of relieving menstrual cramps do not simultaneously address the need to securely and comfortably collect menstrual fluid. Therefore, many women and girls must purchase and use multiple products to address their pain relief and hygiene needs associated with their menstrual period.

### SUMMARY

It would be desirable to provide a device that can relieve menstrual cramps and related pain. It would further be desirable to provide a device that can be used to collect menstrual fluid while relieving menstrual cramps and related pain in a safe, comfortable, convenient, and low-cost manner. Accordingly, the present disclosure provides a heated pain-relieving menstrual cup.

The heated, pain-relieving menstrual cup of the present disclosure is a two-part device comprising a cup body for collecting menstrual fluid and a thermogenic heating ring adapted to securely yet removably attach to the open upper end of the cup body. After the heating ring is activated by the user, the user may simply squeeze the cup body to collapse the device for insertion into the vaginal canal such that the device is positioned with the heating ring at the cervix. The heating ring extends around the cervix when the device is released from the collapsed configuration with the cervix tucked into the cup body. When the device is securely positioned in this manner it provides safe heat to the cervix and uterus, thereby soothing the muscles responsible for menstrual cramps, while securely collecting menstrual fluid. The heating ring provides heat via an exothermic reaction involving a non-toxic composition securely contained within the ring and may be disposable separately from the cup body. In this manner, the heated, pain-relieving menstrual cup of the present disclosure is drug-free, safe, convenient, relatively low-cost, low- to no-maintenance, and easy to use.

The heated menstrual cup includes a main cup body comprising an upper section defining a substantially circular opening and a lower section comprising a fluid collection portion defining a closed lower end; and a flexible ring configured for removable attachment to the upper section of the flexible main cup body at the open upper end and further configured to be positioned around a cervix of a female human, the flexible ring defining a channel entirely enclosed therein. The flexible ring includes a sealed air container positioned within the channel, the air container containing air and comprising a frangible shell constructed to be broken to unseal the air container and release the air into the channel; and a thermogenic composition contained within the channel in isolation from air when the sealed air container is sealed, the thermogenic composition comprising one or more components that generate heat when in contact with the air released from the air container.

In one embodiment, a heated menstrual cup configured to be positioned within a vaginal canal of a female human comprises: a flexible main cup body comprising an upper section defining a substantially circular opening and a lower section comprising a fluid collection portion defining a closed lower end; a removal string attached to the closed lower end; a flexible ring configured for removable attachment to the upper section of the flexible main cup body at the open upper end and further configured to be positioned around a cervix of the female human, the flexible ring defining a channel entirely enclosed therein, wherein the flexible ring comprises: a sealed air container positioned within the channel, the air container defining a compartment containing air and comprising a polymer material, the polymer material being breakable to unseal the air container and release the air into the channel when the flexible ring is biased; and a thermogenic composition contained within the channel in isolation from air when the air container is sealed, wherein the thermogenic composition generates heat when in contact with the air released from the air container when the polymer material is broken.

Various embodiments of the present invention provide one or more such advantages and address, inter alia, the aforementioned issues with known means of relieving menstrual cramps and collecting menstrual fluid.

### BRIEF DESCRIPTION OF THE DRAWINGS

FIG. 1A is a disassembled side perspective view of a menstrual cup according to an embodiment.

FIG. 1B is a cross-sectional view of the menstrual cup of FIG. 1A.

FIG. 1C is an assembled side perspective view of the menstrual cup of FIG. 1A.

FIG. 2A is a side perspective view of the ring of the device of FIG. 1A.

FIG. 2B is a partial cross-sectional view of the ring of the device of FIG. 1A, the cross-section taken parallel to Line A30 of FIG. 2A.

FIG. 3A is another side perspective view of the device of FIG. 1A being manipulated toward a collapsed configuration.

FIG. 3B is a side perspective view of the device of FIG. 1A expanding after having been released from the collapsed configuration.

FIG. 4A is a perspective view of a menstrual cup body according to an alternative embodiment.

FIG. 4B is a top view of the menstrual cup body of FIG. 4A according to an embodiment.

FIG. 4C is a bottom view of the menstrual cup body of FIG. 4A according to an embodiment.

FIG. 5A is a side view of the menstrual cup body of FIG. 4A according to an embodiment.

FIGS. 5B and 5C are cross-sectional views of the menstrual cup body of FIG. 4A according to an embodiment.

FIG. 6A is a perspective view of a heating ring for the menstrual cup body of FIG. 4A according to an embodiment.

FIG. 6B is a top view of the heating ring of FIG. 6A according to an embodiment.

FIG. 6C is a side view of the heating ring of FIG. 6A according to an embodiment.

FIG. 7 is a cross-sectional view of the menstrual cup according to an alternative embodiment.

FIG. 8 is a side view of the device of FIG. 1A positioned within a vaginal canal of a female user such that the ring of the device extends around the user's cervix.

### DEFINITIONS

The term “about” is used here in conjunction with numeric values to include normal variations in measurements as expected by persons skilled in the art, and is understood have the same meaning as “approximately” and to cover a typical margin of error, such as ±5% of the stated value.

Terms such as “a,” “an,” and “the” are not intended to refer to only a singular entity, but include the general class of which a specific example may be used for illustration.

The terms “a,” “an,” and “the” are used interchangeably with the term “at least one.” The phrases “at least one of” and “comprises at least one of” followed by a list refers to any one of the items in the list and any combination of two or more items in the list.

As used here, the term “or” is generally employed in its usual sense including “and/or” unless the content clearly dictates otherwise. The term “and/or” means one or all of the listed elements or a combination of any two or more of the listed elements.

Relative terms such as proximal, distal, left, right, forward, rearward, top, bottom, side, upper, lower, horizontal, vertical, and the like may be used in this disclosure to simplify the description. However, such relative terms do not limit the scope of the invention in any way. Terms such as left, right, forward, rearward, top, bottom, side, upper, lower, horizontal, vertical, and the like are from the perspective observed in the particular figure.

Any direction referred to herein, such as “top,” “bottom,” “left,” “right,” “upper,” “lower,” and other directions or orientations are described herein for clarity and brevity but are not intended to be limiting of an actual device or system. Devices and systems described herein may be used in a number of directions and orientations.

The term “substantially” as used here has the same meaning as “nearly completely,” and can be understood to modify the term that follows by at least about 90%, at least about 95%, or at least about 98%.

The term “elastomer” is used here to refer to a polymer with viscoelasticity (both viscosity and elasticity). Elastomers typically exhibit weak intermolecular forces, low Young's modulus, and high failure strain.

### DETAILED DESCRIPTION

The present disclosure relates to a device that can relieve menstrual cramps and related pain. The present disclosure further relates a device that can be used to collect menstrual fluid while relieving menstrual cramps and related pain in a safe, comfortable, convenient, and low-cost manner. Heated menstrual cups for collecting menstrual fluid and relieving menstrual cramps by providing heat to the muscles responsible for the cramps are provided. Such systems may generally be referred to herein as “menstrual cups” or “heated menstrual cups.”

Compared to known products provided for both collecting menstrual fluid and relieving menstrual cramps, the heated menstrual cups of the present disclosure are drug-free, safer, more convenient, lower cost, lower maintenance, and/or easier to use. The menstrual cups of the present disclosure simultaneously provide secure collection of menstrual fluid and relief from menstrual cramps.

It should be understood that one or more features of the heated menstrual cups described herein may vary from those illustrated and discussed herein, including but not limited to their dimensions, size, and/or proportions relative to other features while remaining within the scope of this disclosure.

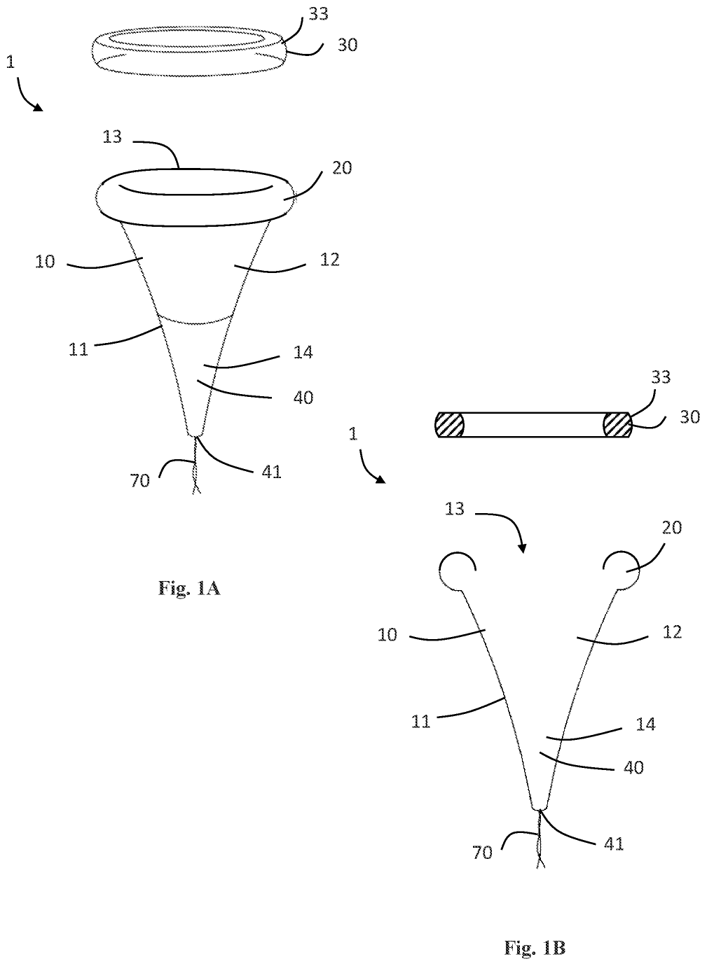

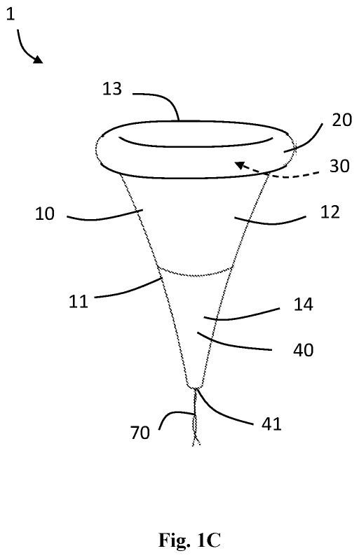

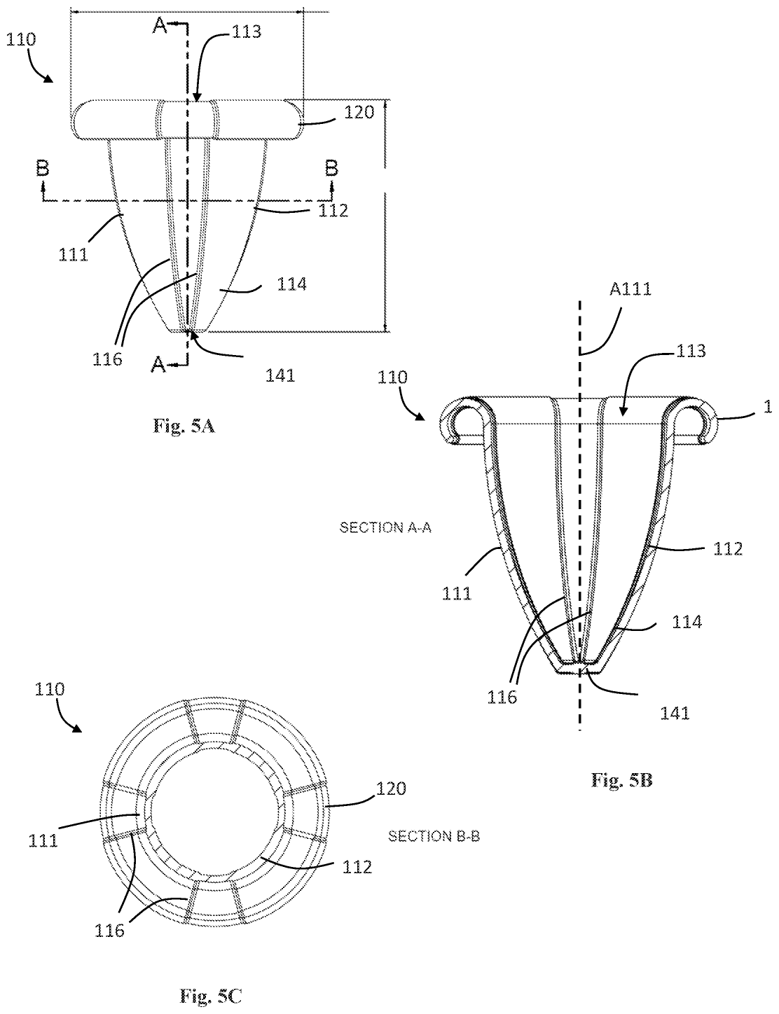

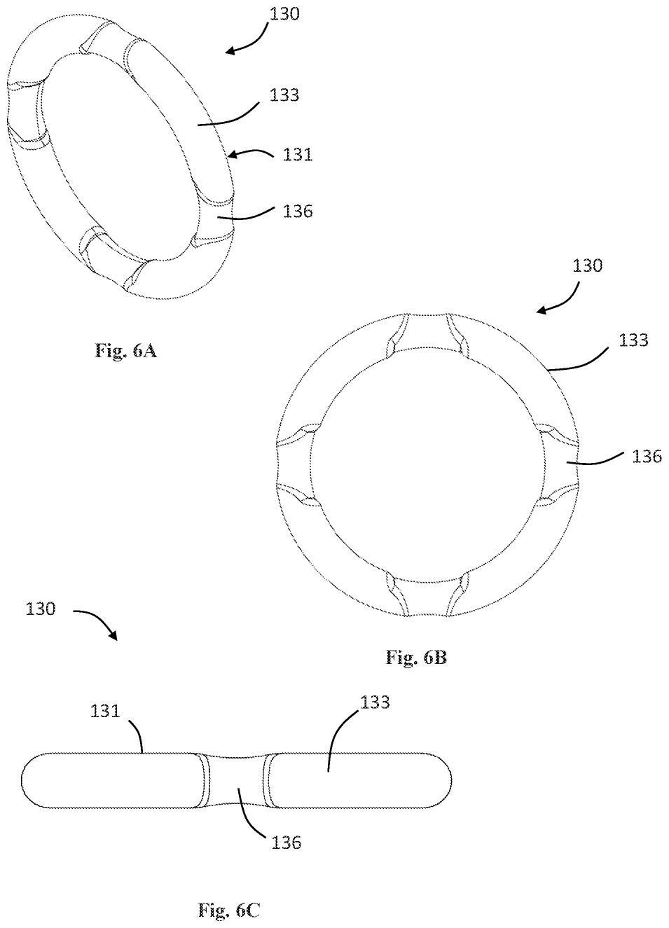

Referring now to FIGS. 1A-1C, an exemplary embodiment of a cramp-relieving and menstrual fluid-collecting cup 1 is shown. According to an embodiment, the menstrual cup 1 has a main cup body 10. The main cup body 10 is hollow and has a cup-shaped wall 11. The cup-shaped wall 11 defines an upper section 12 with an open upper end 13, and a lower section 14. The upper section 12 further defines a substantially circular flange 20. The circular flange 20 may extend (e.g., curve) from the main wall either inward, as shown in the embodiment of FIGS. 1A to 3B, or outward, as shown in the embodiments of FIGS. 5A-6C. A heating ring 30 may be removably coupled with the cup body 10. The heating ring 30 may be removably held in place by the flange 20. The heating ring 30 may define an outer surface 33. The flange 20 may at least partially cover or circumscribe the outer surface 33 of the heating ring 30.

The lower section 14 of the main cup body 10 further defines a fluid collection portion 40. The fluid collection portion 40 has a closed lower end 41. The upper section 12 of the main cup body 10 may have a greater cross-sectional dimension (e.g., diameter) than a cross-sectional dimension of the lower section 14 of the main cup body 10. The cup-shaped wall 11 of the main body 10 may taper from the open upper end 13 to the closed lower end 41. The cup-shaped wall 11 may taper along its entire length or only along a portion of the length. In some embodiments, the cup-shaped wall 11 tapers along the bottom 90%, bottom 80%, bottom 70%, bottom 60%, or bottom 50% of its length. A removal string 70 may be optionally attached to the closed lower end 41 to facilitate removal of the menstrual cup 1 from the vagina.

FIG. 1C is an assembled side perspective view of the menstrual cup 1 of FIG. 1A with the heating ring 30 removably attached to the main cup body 10. As shown in FIG. 1C, the heating ring 30 is configured for placement within the flange 20 of the main cup body 10. In the embodiment shown in FIGS. 1A-1C, the flange 20 extends around the outer perimeter and outer surface 33 of the heating ring 30. The flange 20 covers the top side 31 of the heating ring 30. The flange 20 may extend at least part of the way down along the inner perimeter of the heating ring 30. The heating ring 30 and the flange 20 respectively may be dimensioned such that the heating ring 30 will be securely retained within the flange 20 during insertion, use, and removal of the menstrual cup 1 from the body. For example, the heating ring 30 and the flange 20 may be dimensioned for an interference fit therebetween to securely retain the heating ring 30 to the main cup body 10 until it is removed from the main cup body 10 by a user.

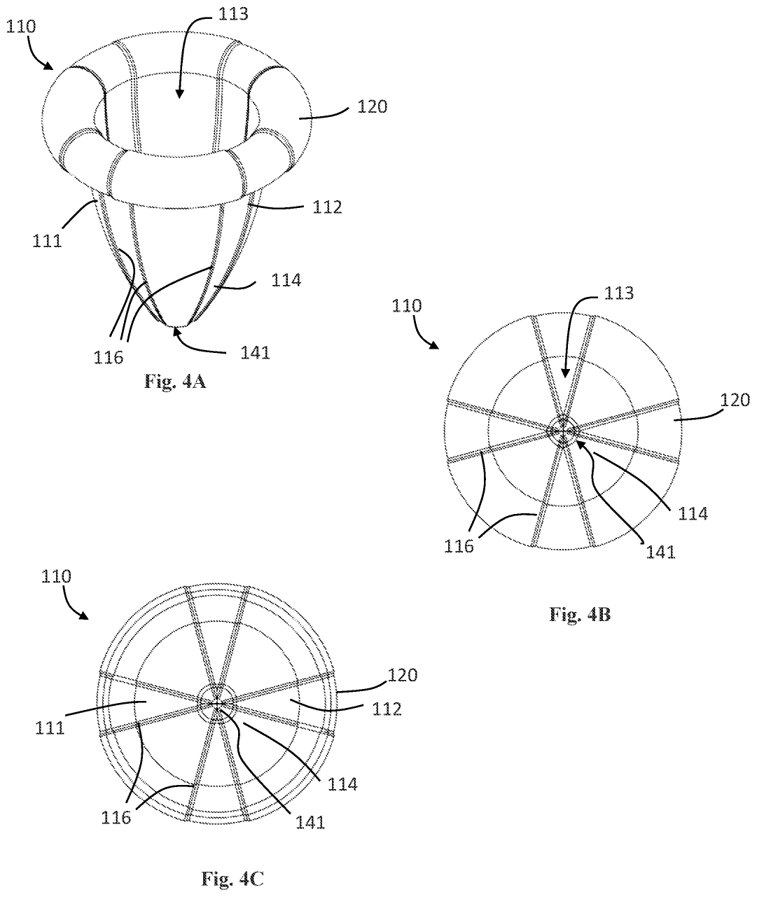

In an alternative embodiment shown in FIGS. 4A-5C, the menstrual cup 100 has a flange 130 that extends outward from the main cup body 110. In this embodiment, the heating ring 130 is disposed on the outside of the cup wall 111. In other respects, the menstrual cup 100 functions substantially similarly to the cup 1 shown in FIGS. 1A-1C. The main cup body 110 is hollow and has a cup-shaped wall 111. The cup-shaped wall 111 defines an upper section 112 with an open upper end 113, and a lower section 114. The upper section 112 further defines a substantially circular flange 120. A heating ring 130 may be removably coupled with the cup body 110. The heating ring 130 may be removably held in place by the flange 120. The heating ring 130 may define an outer surface 133 (see FIG. 6A). The flange 120 may at least partially cover or circumscribe the outer surface 133 of the heating ring 130. In this embodiment, the flange 20 extends around the inner perimeter of the heating ring 130. The flange 120 covers the top side 131 of the heating ring 130. The flange 120 may extend at least part of the way down along the outer perimeter of the heating ring 130. The heating ring 130 and the flange 120 respectively may be dimensioned such that the heating ring 130 will be securely retained within the flange 120 during insertion, use, and removal of the menstrual cup 100 from the body. For example, the heating ring 130 and the flange 120 may be dimensioned for an interference fit therebetween to securely retain the heating ring 130 to the main cup body 110 until it is removed from the main cup body 110 by a user.

The lower section 114 of the main cup body 110 further defines a fluid collection portion 140. The fluid collection portion 140 has a closed lower end 141. The upper section 112 of the main cup body 110 may have a greater cross-sectional dimension (e.g., diameter) than a cross-sectional dimension of the lower section 114 of the main cup body 110. The cup-shaped wall 111 of the main body 110 may taper from the open upper end 113 to the closed lower end 141. The cup-shaped wall 111 may taper along its entire length or only along a portion of the length. In some embodiments, the cup-shaped wall 111 tapers along the bottom 90%, bottom 80%, bottom 70%, bottom 60%, or bottom 50% of its length.

The cup wall 111 may include a plurality of ridges 116 extending longitudinally along the length of the wall 111. The ridges 116 may extend parallel to a longitudinal axis A111 of the cup wall 111. The ridges 116 may facilitate compression of the cup 100 to facilitate insertion and removal. The ridges 116 may extend from the closed lower end 141 to the open upper end 113 of the cup 100. The ridges 116 may further extend along the flange 120. A removal string (not shown) may be optionally attached to the closed lower end 141 to facilitate removal of the menstrual cup 100 from the vagina.

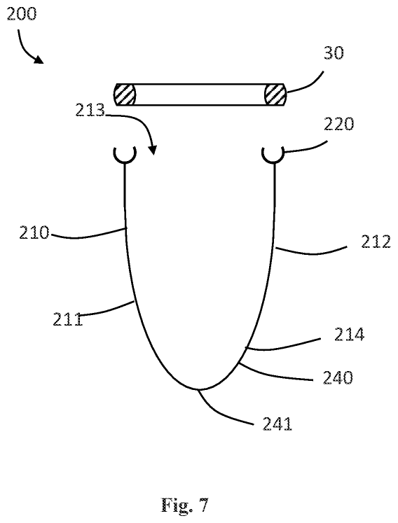

In another alternative embodiment shown in FIG. 7, the menstrual cup 200 has a dual flange 230 that extends upward from the wall 211 of the main cup body 210. The dual flange 230 forms a pocket with an open top constructed to receive the heating ring 30, 130. In other respects, the menstrual cup 200 functions substantially similarly to the cup 1 shown in FIGS. 1A-1C. The main cup body 210 is hollow and has a cup-shaped wall 211. The cup-shaped wall 211 defines an upper section 212 with an open upper end 213, and a lower section 214.

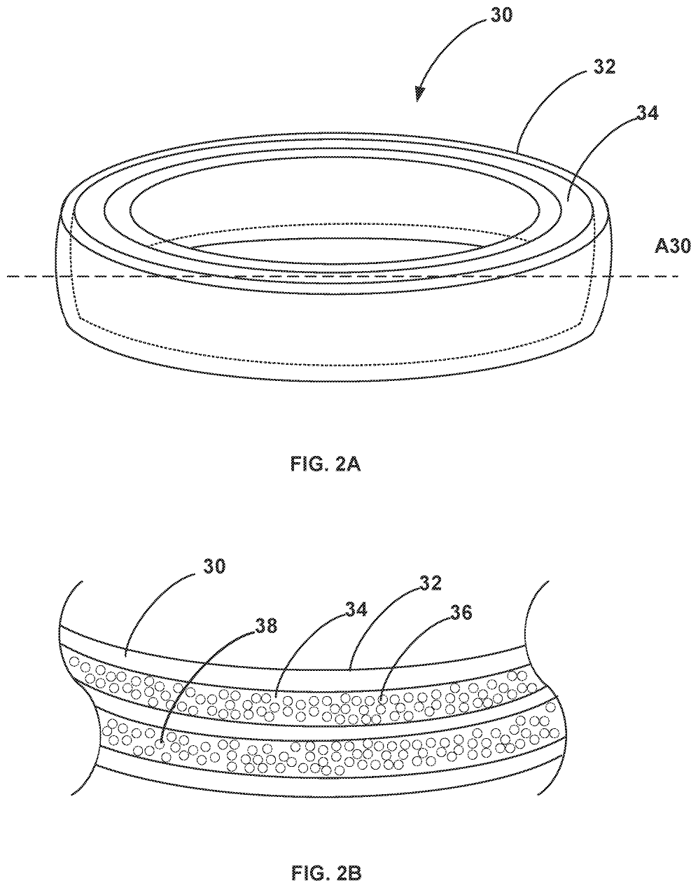

Referring now to FIGS. 2A and 2B, a side perspective view and a schematic, partial cross-sectional view of the heating ring 30 of the menstrual cup 1, the cross-section taken parallel to Line A30 of FIG. 2A, are shown. The heating ring 30 includes a wall 32. The wall 32 defines one or more channels or chambers 34. The one or more channels or chambers 34 are completely enclosed by the wall 32. The wall 32 may define a plurality of channels or chambers 34. The plurality of channels or chambers 34 may be sealed until ruptured by a user. According to an embodiment, the heating ring 30 contains a thermogenic composition 36 in the one or more channels or chambers 34. The thermogenic composition 36 may include multiple components that are separated by the wall 32 in the plurality of chambers 34 until use. The one or more channels or chambers 34 may further contain a sealed container 38 containing oxygen. The sealed container 38 may be contain air. The heating ring 30 may be configured to be used by rupturing the sealed container 38 containing oxygen or a separating wall between chambers 34 and causing the components of the thermogenic composition 36 to mix with the oxygen. A ring with the same internal structure may be used in the menstrual cup 100 of FIGS. 4A-5C as in the cup 1 of FIGS. 1A-1C.

An exemplary heating ring 130 according to an embodiment is shown in FIGS. 6A-6C. The heating ring 130 outer surface 133 may include recessed portions 136. The recessed portions 136 may match the ridges 116 on the cup body 110. The heating ring 130 may include internal chambers 34 as described above with regard to FIGS. 2A and 2B.

The menstrual cup 1, 100 may be made from any suitable materials. According to an embodiment, the menstrual cup 1, 100 is made from a flexible elastomeric material. Preferably the material is selected to have suitable flexibility to facilitate compression of the cup 10, 110 for insertion and removal and sufficient resilience to retain its shape while in use. Preferably, the material is selected to be hypo-allergenic and safe while in use. The material may be selected to be durable to withstand multiple uses. In one embodiment, the main cup body 10, 110 and the heating ring 30, 130 (e.g., the wall 32, 132 of the heating ring 30, 130) may be formed from medical grade silicone. The main cup body 10, 110 and the heating ring 30, 130 (e.g., the wall 32, 132 of the heating ring 30, 130) may be made by manufacturing techniques such as injection molding. Both the main cup body 10, 110 and the heating ring 30, 130 may be washed and re-used if desired. In some embodiments, the main cup body 10, 110 is reusable. In some embodiments, the heating ring 30, 130 is reusable. For example, the heating ring 30, 130 may be washed and re-activated. In some embodiments, the heating ring 30, 130 is configured to provide single-use heat.

According to an embodiment, the thermogenic composition 36 comprises components that are capable of reacting with one another in an exothermic chemical reaction that generates a desired level of heat. Any suitable known thermogenic composition 36 may be used that can be controlled to produce the desired level of heat. A suitable level of heat may heat the heating ring 30, 130 to a temperature of at least 98° F., at least 100° F., at least 102° F., or at least 104° F. The suitable level of heat may be up to 120° F., up to 115° F., up to 110° F., or up to 108° F. The desired level of heat may be achieved by selecting the components of the thermogenic composition 36, by selecting the amounts of the components, and by selecting the positioning of the components within the heating ring 130. In one embodiment, the heating ring 30, 130 contains a source of oxygen. The thermogenic composition 36 includes exothermically reacting components such as iron powder. The thermogenic composition 36 may include water. The thermogenic composition 36 may further include a salt, such as sodium chloride, potassium chloride, or other suitable salt, which may facilitate the reaction between the iron and the oxygen. The thermogenic composition 36 may further include absorbent materials and/or fillers, such as sand, vermiculite, a polymer particulate (e.g., polyacrylate), cellulose, or the like. The thermogenic composition 36 may further include activated charcoal. One or more of the components may be provided as an aqueous dispersion or solution. It should be noted that the thermogenic composition 36 is illustrated in FIG. 2B as a plurality of discrete units for the sake of illustration only. The thermogenic composition 36 may be a powdered mixture, a dispersion, or a solution. The thermogenic composition 36 may contain particles of different sizes. When the components of the thermogenic composition 36 are exposed to oxygen (e.g., oxygen in air), the iron powder reacts with the oxygen in a non-reversible exothermic reaction. Thus, it is desirable to keep the iron isolated from oxygen until the user wishes to activate the heating ring 30, 130. The oxygen for the exothermic reaction may be contained in a sealed air container 38 which may be one of the plurality of channels or chambers 34 of the heating ring 30, 130. While the oxygen (e.g., air) is sealed within the air container 38, the components of the thermogenic composition 36 (e.g., iron) are isolated from oxygen. In order to activate the heating ring 30, 130, the user may bias or squeeze the heating ring 30, 130 to break open the air container 38 and release the oxygen (e.g., air) therein into contact with the thermogenic composition 36 (e.g., iron), allowing the chemical reaction to proceed and produce heat to heat the heating ring 3. The heating ring 30, 130 may be constructed to provide 6 hours or more, 8 hours or more, 10 hours or more, 12 hours or more, 16 hours or more, or 20 hours or more of continuous heat until the oxygen from the air 20 has been consumed. There is no desired upper limit for how long the heating ring 30, 130 may provide heat but in practice, the heating ring 30, 130 may provide heat for up to 24 hours.

The air container 38 may be formed from an air-impermeable polymer material while also sufficiently thin, brittle, or frangible to be easily broken when the heating ring 30, 130 is biased or squeezed by the user, yet strong enough to not be broken during normal transport of the heating ring 30, 130 in its packaging. The air container 38 may be formed of the same material or from a different material than the rest of the heating ring 30, 130. The air container 38 may be formed of a frangible material. The air container 38 may have a frangible shell constructed to be broken to unseal the air container 38. The frangible shell may be made of a polymeric material that is breakable to unseal the air container 38. The material of the air container 38 is also preferably thin enough relative to the wall 32 so as not to pose any risk of broken pieces of the air container 38 puncturing and emerging from the wall 32.

Whenever a user desires to use the menstrual cup with heat, a heating ring 30, 130 may be activated and attached to the main cup body 10, 110. A user may use a new heating ring 30, 130 each time. In this manner, the heated menstrual cups of the present disclosure are low-maintenance and do not require time consuming procedures to re-active a heating material and do not require any electrical heating elements or power sources. In some embodiments, if the user does not desire to use the menstrual cup with heat at a particular time, the heating ring 30, 130 may be attached to the cup body 10, 110 and used without first activating it, or the heating ring 30, 130 may be washed and re-used without heat after previously having been used with heat.

The heated menstrual cups 1, 100 of the present disclosure may be manufactured in a range of different sizes to comfortably accommodate variations in anatomical dimensions among girls and women.

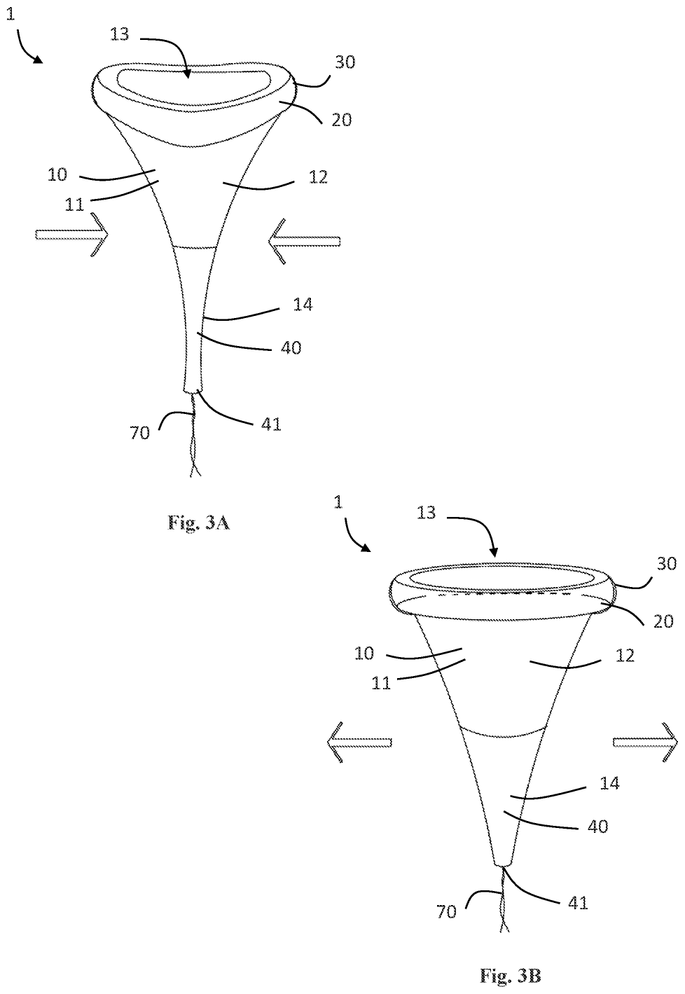

The heated menstrual cup of the present disclosure may be movable between a collapsed configuration, for easy insertion into the vagina, and an open configuration in which the heating ring 30, 130 extends around the user's cervix when the heated menstrual cup is positioned. FIG. 3A is another side perspective view of the heated menstrual cup 1, 100 being manipulated toward the collapsed configuration, with arrows indicating direction of compression. Conveniently, the heated menstrual cup may be manipulated to the collapsed configuration simply by squeezing the main cup body 10, 110 at or around the lower section and mid-section thereof. This causes air to be forced out of the main cup body 10, 110 and the main cup body 10, 110 and heating ring 30, 130 to collapse into a more compact configuration. With the heated menstrual cup 1, 100 in the collapsed configuration, the user may easily insert it into the vagina without the aid of additional applicator tools.

FIG. 3B is another side perspective view of the heated menstrual cup 1, 100 expanding in the direction of the arrows after having been released from the collapsed configuration, such as after the user has inserted the heated menstrual cup into the vagina. The heated menstrual cup 1, 100 thus re-assumes its original form after being positioned in this manner and released.

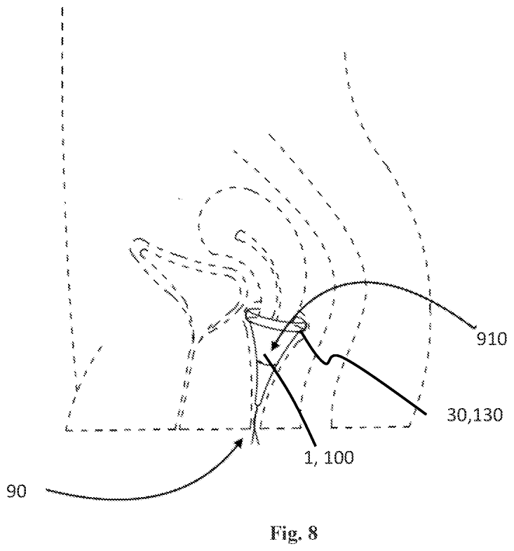

FIG. 8 is a side view of the heated menstrual cup 1, 100 positioned within a vaginal canal 90 of a female user such that the heating ring 30, 130 extends around the user's cervix 910 and the cervix 910 tucks into the main cup body 10, 110. With the heated menstrual cup 1, 100 is in this position, the heating ring 30, 130, if activated, provides long-lasting, continuous heat to the muscles responsible for menstrual cramps while the fluid collection portion of the main cup body 10, 110 securely collects menstrual fluid. The tapered profile of the main cup body 10, 110 makes the heated menstrual cup 1, 100 of this disclosure comfortable to wear while the string 70 enables easy removal. Thus, based on the aforementioned features, the heated menstrual cups 1, 100 of the present disclosure are drug-free, safer, more convenient, lower-cost, lower maintenance, and/or easier to use, than known devices, and simultaneously provide secure collection of menstrual fluid and relief from menstrual cramps.

The description of the invention and its applications as set forth herein is illustrative and is not intended to limit the scope of the invention. Features of various embodiments may be combined with other embodiments within the contemplation of this invention. Variations and modifications of the embodiments disclosed herein are possible, and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

1. A heated menstrual cup comprising:
a main cup body comprising an upper section defining a substantially circular opening and a lower section comprising a fluid collection portion defining a closed lower end; and
a flexible ring configured for removable attachment to the upper section of the flexible main cup body at the open upper end and further configured to be positioned around a cervix of a female human, the flexible ring defining a channel entirely enclosed therein, the flexible ring comprising:
a sealed air container positioned within the channel, the air container containing air and comprising a frangible shell constructed to be broken to unseal the air container and release the air into the channel; and
a thermogenic composition contained within the channel in isolation from air when the sealed air container is sealed, the thermogenic composition comprising one or more components that generate heat when in contact with the air released from the air container.

2. The heated menstrual cup of claim 1, wherein the frangible shell of the sealed air container comprises a polymeric material being breakable to unseal the air container when the flexible ring is biased.

3. The heated menstrual cup of claim 1, wherein the upper section of the main cup body defines a flange configured to extend around an outer surface of the flexible ring.

4. The heated menstrual cup of claim 3, wherein the flange extends inward from the main cup body.

5. The heated menstrual cup of claim 3, wherein the flange extends outward from the main cup body.

6. The heated menstrual cup of claim 1, wherein the main cup body and the flexible ring are collapsible into a collapsed configuration when the lower section is compressed.

7. The heated menstrual cup of claim 6, wherein the main cup body and the flexible ring are configured to return to an expanded configuration when a compressing force is removed from the main cup body.

8. The heated menstrual cup of claim 1, wherein the main cup body and the flexible ring comprise a silicone material.

9. The heated menstrual cup of claim 1, wherein the thermogenic composition comprises iron powder, salt, and water.

10. The heated menstrual cup of claim 9, wherein the thermogenic composition comprises sodium chloride.

11. The heated menstrual cup of claim 9, wherein the thermogenic composition comprises activated charcoal one or more of sand, vermiculite, polymer particles, and cellulose.

12. The heated menstrual cup of claim 1, wherein the upper section of the main cup body has a greater cross-sectional dimension than a cross-sectional dimension of the lower section of the main cup body.

13. The heated menstrual cup of claim 1, wherein the main cup body tapers along at least a portion of its length from the open upper end to the closed lower end.

14. The heated menstrual cup of claim 1, wherein the flexible ring is constructed to generate heat for 6 hours or more upon activation by breaking the frangible shell of the air container.

15. The heated menstrual cup of claim 1, wherein the main cup body comprises a wall defining a longitudinal axis of the menstrual cup and a plurality of ridges extending along the wall parallel to the longitudinal axis.
